(19) United States
(12) Patent Application Publication
(10) Pub. No.: US 2011/0135758 A1
(43) Pub. Date: Jun. 9, 2011

(54) Laxative and process for making same

(76) Inventor: Ruby Thomas, Houston, TX (US)

(21) Appl. No.: 12/592,969

(22) Filed: Dec. 7, 2009

(51) Int. Cl. A61K 33/08; A61P 1/10

(52) U.S. Cl. 424/692

(57) ABSTRACT

A laxative is provided containing a mixture of prune juice, magnesium, magnesium hydroxide and purified water.

### TECHNICAL FIELD

This invention relates to the bodies elimination and cleansing process and more particularly to a laxative composition. Individuals sometimes find it difficult to maintain regularity in the elimination process. Because of this problem a number of things have been tried and developed to assist in this regard. One of the more popular approaches to enhance the elimination process is the consumption of high fiber food, fruits and grains. Additionally, laxatives have been developed to be ingested for this purpose. These laxatives are sometimes made of chemicals, in order to attempt to ensure that regularity is achieved. Some of the laxatives that have been developed to achieve the desired result have become very extravagant mixtures of many different ingredients, which are sometimes harsh and cannot be used regularly. Additionally the different ingredients in these mixtures are not always readily available. Accordingly, there becomes a need to provide a laxative which, when used, will consistently provide the desired result while at the same time being preparable by utilizing readily available ingredients which are not harsh and can be used regularly.

### BACKGROUND ARTS

Attempts have been made to provide laxatives which provide some regularity. One such laxative is disclosed in U.S. Pat. No. 5,753,232. This patent discloses an ingestible syrup. This composition, softens the stool and promotes elimination regularity. The composition includes from about 90 to 99 volume % prune concentrate and from about 10 to 1 volume % psyllium powder. The mixture is a stable, pourable syrup that may be easily consumed.

Another laxative product is disclosed in U.S. Pat. No. 5,223,298. This invention relates to a novel breakfast cereal which contains a quantity of psyllium sufficient to provide a hypocholesterolemic effect if ingested. The source for psyllium can be psyllium seed husk, whole psyllium seed or dehusked psyllium seed. When ingested, the psyllium containing cereal also effectively acts as a laxative.

Although both these products achieve the desired results, both these products because of the unique ingredients required may be expensive and somewhat difficult to make. Accordingly it is desirable to provide a product made from readily available ingredients, which is easy to prepare and has the unique qualities and advantages of the laxative of the present invention.

### DISCLOSURE OF THE INVENTION

The invention relates to a laxative. The laxative in accordance with the principles of the invention is provided in predetermined weight quantities with a mixture of prune juice, magnesium, magnesium hydroxide and purified water.

### BEST MODE FOR CARRYING OUT THE INVENTION

In accordance with the preferred embodiment of the invention, a novel laxative is provided. The laxative in accordance with the present invention contains on a weight basis 78% prune juice, 1.2% magnesium, 2.8% magnesium hydroxide and 18% purified water.

A typical process for preparation of the laxative includes a first step of mixing the prune juice, magnesium, magnesium hydroxide, and purified water at ambient temperature. Once these ingredients are mixed together they are warmed over a low temperature between 100 degrees F. and 150 degrees F. for approximately 3 minutes or until the ingredients form a homogenous mixture. The resulting mixture is the laxative of this invention.

The laxative is then cooled to ambient temperature and appropriately can be packaged in a hard thick plastic container or other such self heating package as desired.

When it is desired to take the laxative the laxative is again warmed and then ingested by the user. This can be followed by the ingestion of 6 to 8 ounces of spring water. The result is a quick stimulant which produces a quick evacuation of the bowel.

The invention has been shown and described in what is considered to be the most practical and preferred embodiment. However, it should be recognized that changes may be made by those skilled in the art without departing from the spirit and scope of the invention.

What is claimed:

1. A process for making a laxative consisting of:
blending together laxative ingredients including by weight 78% prune juice, 1.2% magnesium, 2.8% magnesium hydroxide and 18% purified water at a first predetermined temperature; and
warming these ingredients at a second predetermined temperature for a predetermined time to form a homogenous mixture;
cooling the mixture to the first predetermined temperature; and
packaging the mixture in a container.

2. A process as defined in claim 1 wherein the first predetermined temperature is ambient temperature.

3. A process as defined in claim 2 wherein the second predetermined temperature is between 100 degrees F. and 150 degrees F.

4. A process as defined in claim 3 wherein the predetermined time is three minutes.

5. A laxative product made by the process of claim 1.

6. A laxative product made by the process of claim 2.

7. A laxative product made by the process of claim 3.

8. A laxative product made by the process of claim 4.
